(19) United States
(12) Patent Application Publication
(10) Pub. No.: US 2012/0302516 A1
(43) Pub. Date: Nov. 29, 2012

(54) NANOPARTICLES FOR DRUG DELIVERY

(75) Inventors: Michael H. Nantz, Louisville, KY (US); Souvik Biswas, Louisville, KY (US)

(73) Assignee: UNIVERSITY OF LOUISVILLE RESEARCH FOUNDATION, INC., Louisville, KY (US)

(21) Appl. No.: 13/502,941

(22) PCT Filed: Oct. 19, 2010
(86) PCT No.: PCT/US10/53235

§ 371 Date: Aug. 8, 2012

Related U.S. Application Data
(60) Provisional application No. 61/252,934, filed on Oct. 19, 2009.

(51) Int. Cl. C07H 15/252; C07H 23/00; C07H 1/00; C07C 239/16; C07C 249/08; A61K 31/295; G01N 33/53; C07C 45/79; A61N 2/10; C07C 251/58; A61K 31/704; B82Y 40/00; B82Y 5/00

(52) U.S. Cl. 514/34; 536/6.4; 556/37; 536/55; 556/138; 514/502; 436/501; 435/7.1; 568/303; 568/420; 600/12; 977/773; 977/906; 977/896

(57) ABSTRACT

The invention provides magnetic nanoparticles comprising a core, wherein the nanoparticles comprise at least one therapeutic agent linked to the core via a hydrazone linkage or via an oxime ether linkage, methods for making said nanoparticles, and methods for using said nanoparticles.

### CROSS-REFERENCE TO RELATED APPLICATION(S)

This patent application claims the benefit of priority of U.S. application Ser. No. 61/252,934, filed Oct. 19, 2009, which application is herein incorporated by reference.

### STATEMENT OF GOVERNMENT SUPPORT

This invention was made with government support under Grant number R21DE029271 awarded by the National Institutes of Health. The government has certain rights in the invention.

### BACKGROUND

An important attribute of a drug delivery system is its ability to allow for regulated drug release, thereby minimizing side effects and improving therapeutic efficacy of conventional pharmaceuticals. Different approaches can be used to regulate the release of the therapeutic payload from the carrier. For example, endogenous strategies exploit specific physiochemical characteristics of the physiological microenvironment, providing biologically controlled release. Exogenous strategies provide a complementary approach, employing external stimuli to cause controlled drug release. Ideally, a drug delivery system would allow for spatiotemporal regulated release of the drug. Currently, there is a need for a drug delivery system that would provide drug release that could be targeted spatially, temporally, or both spatially and temporally.

### SUMMARY OF CERTAIN EMBODIMENTS

Accordingly, certain embodiments of the present invention provide a magnetic nanoparticle comprising a core, wherein the nanoparticle comprises at least one therapeutic agent linked to the core via a hydrazone linkage or via an oxime ether linkage.

Certain embodiments of the present invention provide a magnetic nanoparticle comprising a core, wherein the nanoparticle comprises reactive hydrazine or aminooxy groups linked to the core of the nanoparticle.

In certain embodiments, the core of the nanoparticle is about 5-50 nm (e.g., about 10-15 nm) in diameter. In certain embodiments, the size of the nanoparticle is of appropriate size to heat in vivo with an alternating electromagnetic field to release a therapeutic agent.

In certain embodiments, at least one therapeutic agent is a chemotherapeutic agent, an antibiotic agent, an antifungal agent, an antiparasitic agent or an antiviral agent.

In certain embodiments, at least one therapeutic agent is a chemotherapeutic agent.

In certain embodiments, at least one therapeutic agent is an anthracycline antibiotic.

In certain embodiments, at least one therapeutic agent is doxorubicin.

In certain embodiments, the nanoparticle has an iron oxide core.

In certain embodiments, at least one therapeutic agent is linked to the core via a hydrazone linkage.

In certain embodiments, at least one therapeutic agent is linked to the core via an oxime ether linkage.

In certain embodiments, the nanoparticle comprises reactive hydrazine groups linked to the core of the nanoparticle.

In certain embodiments, the nanoparticle comprises reactive aminooxy groups linked to the core of the nanoparticle.

In certain embodiments, carbohydrates or carbohydrate fragments may be used to anchor the aminooxy reagent to the nanoparticle.

In certain embodiments, the nanoparticle further comprises a targeting element.

In certain embodiments, the nanoparticle further comprises a carbohydrate or carbohydrate fragment.

Certain embodiments of the present invention provide a method of making a nanoparticle, comprising combining a magnetic nanoparticle having a core with aminooxy agent, e.g., ammonium or aminium aminooxy agent, to make an iron oxide nanoparticle that comprises reactive aminooxy groups linked to the core of the nanoparticle.

In certain embodiments, the method further comprises reacting the nanoparticle that comprises the aminooxy groups with at least one agent (e.g., an agent having a reactive aldehyde or ketone group) to make a nanoparticle that comprises at least one agent linked to the core of the nanoparticle via an oxime ether or hydrazone linkage.

In certain embodiments, at least one agent is a therapeutic agent.

In certain embodiments, the aminooxy agent is an agent having the formula:

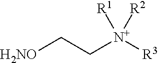

wherein R¹, R², and R³ are each individually alkyl optionally substituted with one or more —OH, —CF₃, —N⁺, or —ONH₂ groups.

In certain embodiments, the aminooxy agent is a compound selected from

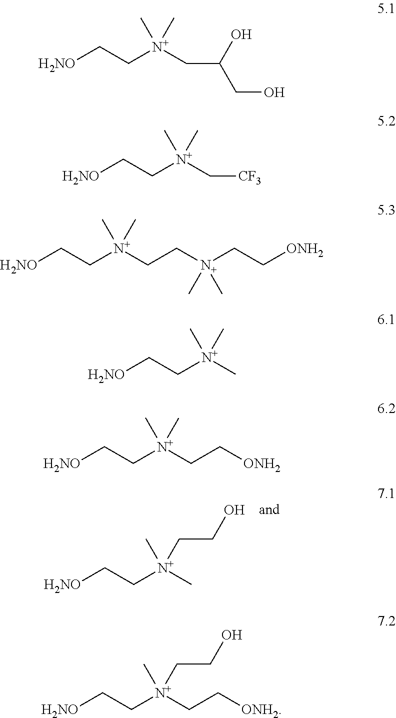

Certain embodiments of the present invention provide nanoparticles made according to the methods described herein.

Certain embodiments of the present invention provide a method for administering a therapeutic agent to a patient, comprising administering a nanoparticle as described herein to the patient.

In certain embodiments, the method further comprises delivering a source of heat so as to release the therapeutic agent from the nanoparticle.

In certain embodiments, an alternating electromagnetic field is used to release the therapeutic agent from the nanoparticle.

In certain embodiments, the method further comprises magnetically targeting the nanoparticles to a specific location in the patient.

In certain embodiments, the nanoparticle comprises a targeting element.

Certain embodiments of the present invention provide a method for separating a compound having a reactive aldehyde or ketone group from a mixture of compounds, comprising:

adding a nanoparticle described herein to the mixture;

allowing the nanoparticle to bind to the compound having a reactive aldehyde or ketone group; and

separating the bound nanoparticle from the mixture.

In certain embodiments, the method further comprises identifying the compound bound to the nanoparticle.

Certain embodiments of the present invention provide a method for administering a therapeutic agent to a patient, comprising:

administering a nanoparticle as described herein to the patient;

targeting the nanoparticle to a specific site in the patient's body;

administering a therapeutic agent that comprises an aldehyde or ketone group to the patient;

allowing the nanoparticle and therapeutic agent to bind together; and

applying an alternating electromagnetic field to the specific site in the patient's body to release the therapeutic agent from the nanoparticle.

In certain embodiments, the nanoparticle is targeted to the specific site magnetically.

In certain embodiments, the nanoparticle comprises a targeting element that targets the nanoparticle to the specific site.

Certain embodiments of the present invention provide a composition comprising a nanoparticle as described herein and an acceptable carrier.

In certain embodiments, the acceptable carrier is a pharmaceutically acceptable carrier.

In certain embodiments, the composition comprises a first population of nanoparticles that are individually linked via a hydrazone linkage or an oxime ether linkage to a first therapeutic agent and a second population of nanoparticles that are individually linked via a hydrazone linkage or an oxime ether linkage to a second therapeutic agent that is a different therapeutic agent than the first therapeutic agent.

Certain embodiments of the present invention provide a nanoparticle as described herein for use in medical treatment or diagnosis.

Certain embodiments of the present invention provide the use of a nanoparticle as described herein to prepare a medicament useful for treating cancer in an animal (e.g., cancers, such as bladder, breast, head and neck, liver, lung, ovary, pancreas, prostate, thyroid and uterus cancer, e.g., breast cancer).

Certain embodiments of the present invention provide a nanoparticle as described herein for use in therapy.

Certain embodiments of the present invention provide the use of a nanoparticle as described herein for treating cancer.

Also contemplated is a method that comprises delivering a first agent using the nanoparticles described herein, wherein the first agent activates a second agent, which second agent has be delivered, e.g., to a specific site in a patient's body, using, e.g., conventional means. Thus, the second agent is activated only at the site of heat treatment (i.e., the site where the primary agent was released from the nanoparticle).

In certain embodiments, the nanoparticles are useful for acute treatment and for treatment at a specific site and not for prolonged or systemic treatment.

### DETAILED DESCRIPTION

Functionalized Nanoparticles for Magnetically-Guided, Heat-Induced Drug Delivery

As described herein, it has been demonstrated that magnetic nanoparticles can be modified, e.g., with aldehydes or ketones. A therapeutic agent (i.e., a “drug”) can be attached to the nanoparticle surface via a hydrazone or oxime ether linkage. Similarly, a targeting element can also be attached, e.g., in combination with the therapeutic agent, to the nanoparticle. The nanoparticle-drug (NP-D) formulation is stable under aqueous, physiological conditions. However, when the NP-D formulation is heated, the drug is released as an oxime ether conjugate. Oxime ether conjugates are a well-known class of pro-drugs, and several pharmaceutically active agents are administered as oxime ether analogs. NP-D formulations have been heated using an oil bath warmed to 45° C., and these experiments showed that a thermal stimulus causes compound release. The NP-D formulation also releases the compound on exposure to an alternating electromagnetic field (AEM field; see, e.g., Tang et al., Biomaterials, 29, 2673-2679 (2008)). It is believed that the nanoparticles are heated by application of sources of energy to cause the release. Accordingly, any source of energy that causes the release (presumably by heating the individual nanoparticles), is suitable for use.

It will also be possible to target the nanoparticles to a specific location in a patient's body, e.g., by magnetically guiding the nanoparticles to the target tissue and/or by conjugating appropriate targeting elements (e.g., an antibody fragment, a small molecule ligand of a cellular receptor) to the NP-D formulation using, e.g., the established oxime ether approach.

Thus, functionalized magnetic nanoparticles, e.g., iron oxide nanoparticles, can be reacted with a pharmaceutical agent containing, e g., an aldehyde or ketone group, and optionally conjugated with a targeting element. The ‘loaded’ nanoparticles can be administered to a patient and the drug released on exposure, e.g., to a stimulus sufficient to cause release of the drug (e.g., a focused, externally-applied stimulus e.g., an AEM field). In certain embodiments, the nanoparticles can be magnetically guided to the desired location in the body of the patient. In certain embodiments, the ‘loaded’ nanoparticles are biologically inactive, e.g., with respect to the drug. This delivery system provides a method for delivering drugs that are toxic when administered systemically by allowing for targeting of the drug to a specific location. Thus, this system is particularly useful for delivering drugs that are beneficially delivered to a specific location at a high concentration, e.g., anticancer, antibiotic, antifungal, antiparasitic, and antiviral drugs. An advantage of this delivery is to limit the systemic exposure to the drug while targeting the delivery of the drug to a selected location.

The aminooxy nanoparticles are also useful as reagents for analytical work. For example, a cell lysate treated with the aminooxy nanoparticles would be expected to scavenge the aldehyde and ketone metabolites from the biological milieu. Separation, e.g., magnetic separation, could then be performed to remove the nanoparticles from the lysate mixture. Subsequent heating would release the bound ketones and aldehydes, and analysis of the supernatant would give a profile of only those metabolites.

For example, iron oxide nanoparticles (>95% Fe₃O₄ magnetite, about 10-15 nm diameter) are prepared such that they retain an overall negative charge (zeta potential in H₂O, −32 mV). These magnetic nanoparticles are coated with tetraalkylammonium salts (R₄N⁺) by a simple mixing procedure. The electrostatic interactions between oxide and ammonium ions, as well as hydrogen bonding interactions between oxide and resident polar functionality in the R₄N⁺ species, facilitate the loading and retention of the ammonium salts on the surface of the nanoparticles. The tetraalkylammonium salts can contain chemical functional groups for binding, e.g., covalently binding, pharmaceutical agents, such as aminooxy (RONH₂) or hydrazine (RNHNH₂) moieties. In this way, a drug can be bound via an ammonium salt ‘prodrug’ form to magnetic iron oxide nanoparticles. In certain embodiments the NP-D formulation is pharmaceutically inactive.

Specifically, the aminooxy-functionalized (R—ONH₂) ammonium salts 1, 2, 3 and 4 below have been prepared. The nanoparticles (NP) were coated with compound 1 as a representative example to form aminooxy-functionalized nanoparticles NP.1 and then reacted with sample aldehydes to obtain the NP.1.drug adducts. The aldehyde is thus bound to the magnetic nanoparticle delivery vehicle via an oxime ether linkage (—ON═CHR). Oxime ethers have been used to derivatize aldehyde or ketone-based drugs, so this is a recognized prodrug form that liberates its drug on exposure to acidic conditions (oxime ether hydrolysis), such as those found within endosomes or external to tumors.

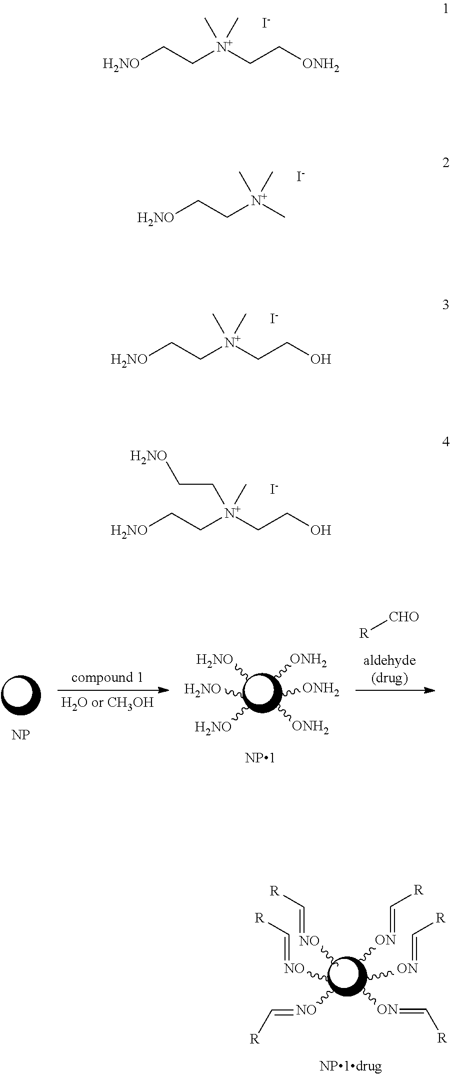

While the NP.1.drug complex could be used to deliver its bound drug via a conventional pro-drug hydrolysis mechanism after delivery to target tissue (e.g., magnetically guided), another potentially more useful method for drug release was discovered. It has been discovered that briefly warming the nanoparticle complex to 42° C. resulted in separation of the bound conjugate from the nanoparticle. Since magnetic, e.g., iron oxide, nanoparticles embedded within tissue can be readily heated to temperatures as high as 45° C. by exposure to an alternating electromagnetic field (a technique used in thermotherapy of cancer, thermoablation), the complex NP.1.drug can be warmed in similar manner to release its payload. Thus, a magnetic nanoparticle delivery system has been developed that is capable of binding aldehyde or ketone substrates and releasing these substrates in response to a heat stimulus, e.g., a heat stimulus applied using an externally focused source. Spatial and temporal control over drug release (e.g., the drug is released at site of electromagnetic field irradiation at a specific time) is particularly appealing for targeted delivery applications, especially if NP.1.drug is pharmaceutically inactive.

The nanoparticles could be used to deliver an aldehyde- or ketone-based drug (or an aldehyde- or ketone-modified analog of a drug) that may otherwise be too toxic for conventional delivery. Loading the drug onto nanoparticles would ameliorate the cytotoxic effects until the drug is released at the site where an electromagnetic field is applied, e.g., for use in delivering chemotherapeutic drugs. Thus, in certain embodiments, the drug is an aldehyde- or ketone-based drug and in certain embodiments the drug is an aldehyde- or ketone-modified analog of a drug.

Aldehydes and ketones are common functional groups in organic chemistry. In certain embodiments, aldehyde- or keto-analogs of drugs are prepared. For example, a drug possessing a carboxylic acid group can be converted into an amide derivative that features an aldehyde group (e.g., RCO₂H→RC(O)NHCH₂CH₂CHO). Alcohol-based drugs in which the alcohol moiety is not essential for pharmaceutical activity can be oxidized to provide an aldehyde or ketone for NP conjugation.

The magnetic properties of the nanoparticle system could also be exploited to improve localization of a drug in a target tissue by using an externally applied magnetic field followed by irradiation to release the drug, e.g., for use in magnetically guided drug delivery.

The mechanism of heat-induced ammonium salt disassociation from the iron oxide nanoparticle is unique. Other drug delivery systems typically rely on hydrolyses of chemical linkages to release the drug, or on the dissolution of a matrix that encapsulates the drug. These drug release mechanisms are difficult to control. As described herein, an externally applied electromagnetic field could be used to control where drug release occurs. Nanoparticle complexes elsewhere would be cleared (e.g., by the kidney) without causing effects, especially when the complex itself is pharmaceutically inactive.

The term “alkyl” as used herein refers to alkyl groups having from 1 to 10 carbon atoms, which are straight or branched monovalent groups.

### Administration

The method of administering the nanoparticles to the desired area for treatment and the dosage may depend upon, but is not limited to, the type and location of the disease material. The size range of the nanoparticles may allow for microfiltration for sterilization. Some methods of administration include intravascular injection, intravenous injection, intraperitoneal injection, subcutaneous injection, and intramuscular injection. The nanoparticles may be formulated in an injectable format (e.g., suspension, emulsion) in a medium such as, for example, water, saline, Ringer's solution, dextrose, albumin solution, and oils. The nanoparticles may also be administered to the patient through topical application via a salve or lotion, transdermally through a patch, orally ingested as a pill or capsule or suspended in a liquid or rectally inserted in suppository form. Nanoparticles may also be suspended in an aerosol or pre-aerosol formulation suitable for inhalation via the mouth or nose. Once administered to the patient, delivery of the nanoparticles to the target site may be assisted by an applied static magnetic field due to the magnetic nature of the nanoparticles. Assisted delivery may depend on the location of the targeted tissue. The nanoparticles may also be delivered to the patient using other methods. For example, the nanoparticles may be administered to the patient orally, or may be administered rectally.

The invention will now be illustrated by the following non-limiting Examples.

### EXAMPLE 1

### Synthesis of Representative Tetraalkylammonium Aminooxy Reagents

Described herein is the synthesis of representative aminooxy reagents useful, e.g., in preparing the nanoparticles described herein. Further, certain reagents may contain —OH groups, e.g., multiple —OH groups, (as in 5.1 below). In certain embodiments, carbohydrates or carbohydrate fragments may be used to anchor the aminooxy reagent to the nanoparticle. In certain embodiments, electron-withdrawing groups, such as —CF₃ (as in 5.2 below) can be used to increase the effective positive charge of the ammonium ion to more strongly anchor the reagent. In certain embodiments, aminooxy reagents with multiple ammonium ions (as in 5.3 below) can be used to improve association with the iron oxide.

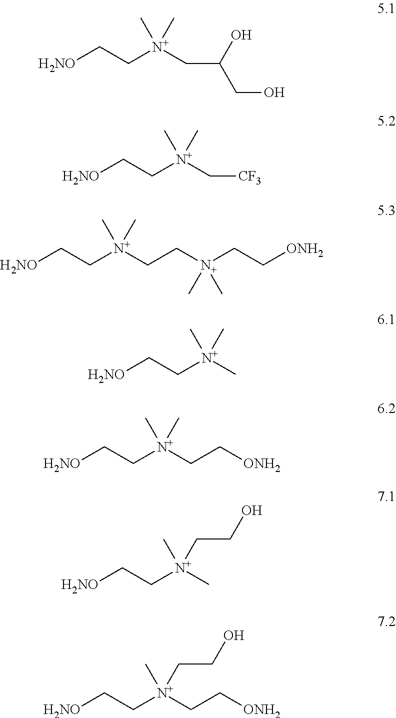

In certain embodiments, the reagent has the formula:

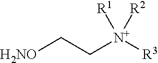

wherein R¹, R², and R³ are each individually alkyl optionally substituted with one or more —OH, —CF₃, —N⁺, —ONH₂ groups. It should be noted that the linker connecting the —ONH₂ may also be alkyl. In certain embodiments, R¹, R², and R³ may be optionally substituted with an electron-withdrawing group.

In certain embodiments, the reagent comprises polyhydroxyl groups. In certain embodiments, the reagent comprises —C(H₂O)—H. In certain embodiments, electron-withdrawing groups can be included to increase the N+ effectiveness and create tighter associations with the iron oxide surface, as shown in: Nantz et al., Biochimica et Biophysica ActaI 1998, 1394, 219-223. In certain embodiments, bis(ammonium) salts are used.

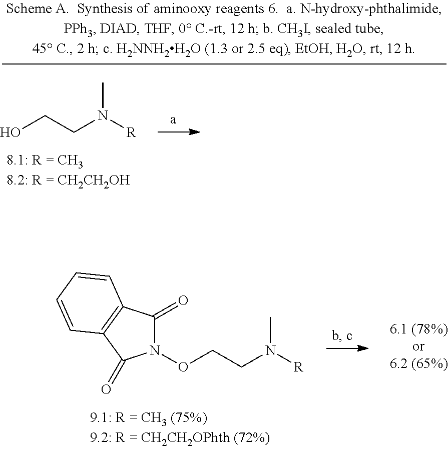

Reaction of the commercially available ethanolamines 8.1 and 8.2 (Scheme A) with N-hydroxypthalimide (NHP) under Mitsunobu conditions (Grochowski et al., Synthesis 1976, 682-684) (equimolar amounts of NHP:PPh₃:DIAD) furnished phthaloyloxy amines 9.1 and 9.2, respectively, in good yields. Amine quaternization was best accomplished by gently warming the amines in methyl iodide (ca. 0.2 M). The resultant, crude ammonium iodides were treated directly with hydrazine in ethanol to cleave the phthaloyl groups. After work-up, the water-soluble aminooxy reagents 6.1 and 6.2 were isolated by lyophilization of the aqueous layer and then purified using reverse phase HPLC.

By analogy to previous syntheses of N-(2-hydroxyethyl)ammonium salts, it was expected that the N-alkylation of 9.1 using 2-bromoethanol would be a convenient route to hydroxyethyl-functionalized reagent 7.1 (Scheme B). However, the N-alkylation required heating the reactants at 60° C., and this resulted in a complex mixture of products containing ammonium bromide 10.1. Subsequent hydrazinolysis failed to deliver a product mixture that was more amenable to purification. Consequently, 7.1 was obtained in only poor yields (ca. <20%). Due to these complications, N-methylation was relied on as the penultimate, ammonium salt-forming step.

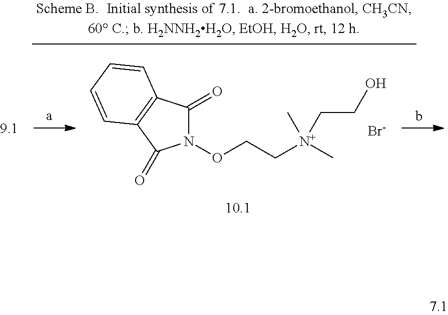

Monosilylation of di-(8.2) and triethanolamine (14.2, Scheme C) was accomplished by reacting an excess of each ethanolamine with TBSCl as the limiting reagent. The resultant, mono-protected ethanolamines 11.1 and 11.2 were then transformed to the corresponding N-(2-hydroxyethyl)-functionalized aminooxy reagents using the path established for synthesis of reagents 6. While desilylation of the more polar phthaloyloxy amine 12.1 proceeded smoothly on work-up by stirring with aq. HCl, this approach did not work for phthaloyloxy amine 12.2. Furthermore, standard TBAF-mediated deprotection of 12.2 resulted in double N—O cleavage, giving 14.2 as the principal product. Other attempts (e.g., AcOH- or TsOH-mediated deprotections) were equally disappointing. However, prolonged reaction with aqueous HF furnished the desired product 13.2. The amine quaternizations, this time using methyl iodide, and subsequent hydrazinolyses proceeded without incident, and the cationic aminooxy reagents 7.1 and 7.2 were isolated in good overall yield.

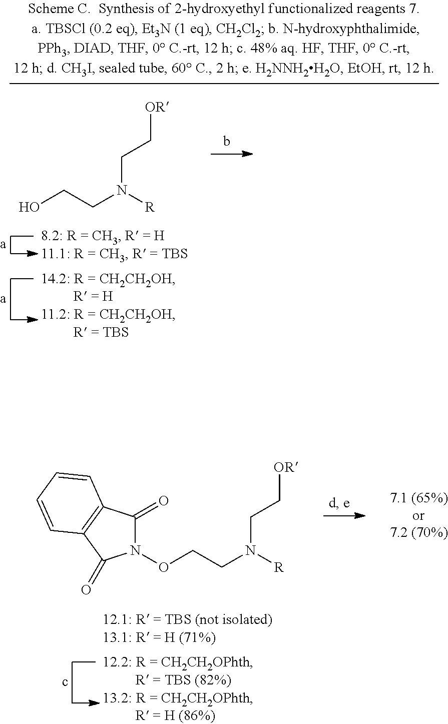

N-(2-(aminooxy)ethyl)-N,N,N-trimethylammonium iodide (6.1). To a solution of triphenylphosphine (15.3 g, 58.3 mmol) and N-hydroxyphthalimide (9.50 g, 58.3 mmol) in THF (200 mL) at 0° C. was added dropwise N,N-dimethylethanolamine (8.1) (4.33 g, 48.6 mmol). After stirring 30 min, diisopropyl azodicarboxylate (DIAD) (11.5 mL, 58.3 mmol) was added slowly via syringe. The reaction mixture was stirred an additional 30 min at 0° C. and then allowed to warm to room temperature. After 12 h, the solvent was removed by rotary evaporation. EtOAc (150 mL) was added to dissolve the residue followed by successive washings with saturated aq. NaHCO₃ (3×100 mL), water (50 mL), and brine (3×100 mL). The organic layer then was dried (Na₂SO₄), filtered, and concentrated to ˜50 mL by rotary evaporation. The organic layer was cooled using an ice bath and cold 5% aq. HCl (30 mL) was added. On complete addition, the mixture was warmed to room temperature and stirred 20 min. The aqueous layer was separated, washed with Et₂O several times, cooled to 0° C., and then made alkaline (not to exceed pH 8) by slowly adding saturated aq. NaHCO₃. The alkaline aqueous layer was extracted using chloroform (3×50 mL). The combined organic phase was dried (Na₂SO₄), filtered, and the solvent removed by rotary evaporation to afford aminooxy phthalimide 9.1 (8.53 g, 75%) as a light yellow solid which required no further purification for use in the next step; ¹H NMR (DMSO-d₆, 500 MHz) δ 7.82 (d, J=5.0 Hz, 4H), 4.21 (t, J=5.0 Hz, 2H), 2.60 (t, J=5.0 Hz, 2H), 2.17 (s, 6H); ¹³C NMR (DMSO-d₆) δ 163.74, 135.35, 129.25, 123.80, 76.08, 57.45, 45.86.

9.1 (0.50 g, 2.13 mmol) was placed in a sealed tube and iodomethane (6.5 mL) was added. The mixture was degassed using a stream of nitrogen and then the tube was sealed and warmed to 50° C. After 2 h, the sealed tube was cooled, opened, and the solvent was evaporated (caution: fume hood required) to afford the crude ammonium iodide as a light yellow solid; ¹H NMR (DMSO-d₆, 500 MHz) δ 7.89 (s, 4H), 4.67 (br s, 2H), 3.8 (t, J=5.0 Hz, 2H), 3.24 (s, 9H); ¹³C NMR (DMSO-d₆) δ 164.01, 135.89, 129.45, 124.33, 72.32, 64.19, 54.16.

The crude iodide salt was dissolved in a mixture of EtOH (5 mL) and H₂O (0.05 mL) Hydrazine monohydrate (1.13 mL, 14.9 mmol) was added and the reaction mixture was stirred 4 h at room temperature. The solvents were removed by rotary evaporation and the residue was dissolved in H₂O (15 mL). The aqueous solution was extracted several times with EtOAc and then the H₂O was evaporated by freeze drying to yield aminooxy 6.1 (0.41 g, 78%) as a light yellow amorphous solid; ¹H NMR (DMSO-d₆, 500 MHz) δ 6.23 (s, 2H), 3.91 (br s, 2H), 3.52 (br s, 2H), 3.07 (s, 9H); ¹³C NMR (DMSO-d₆) δ 69.3, 64.1, 54.0. HPLC analysis (Atlantis C₁₈ 5 μm column, gradient elution using 100% H₂O to 100% CH₃CN over 10 min at a flow rate of 1 mL/min) indicated excellent sample homogeneity (98% purity, tᵣ=1.88 min). The elution profile was monitored by UV absorbance at 214 nm. HRMS calcd for C₅H₁₅N₂O⁺ (M⁺) 119.1179, found 119.1178.

N-(2-(aminooxy)ethyl)-N-(2-hydroxyethyl)-N,N-dimethylammonium iodide (6.2). Using the procedure described for synthesis of 6.1, N-methyl-diethanolamine (8.2) (2.0 g, 16.8 mmol) was transformed into the corresponding bis-(phthaloyloxyethyl)amine 9.2 (4.94 g, 72%); ¹H NMR (DMSO-d₆, 500 MHz) δ 7.81 (s, 8H), 4.18-4.21 (m, 4H), 2.78-2.81 (m, 4H), 2.30 (s, 3H); ¹³C NMR (DMSO-d₆) δ 164.0, 135.6, 129.5, 124.1, 76.4, 55.9, 43.0. A portion of this material (0.50 g, 1.22 mmol) then was treated with iodomethane in the manner described above to afford the corresponding ammonium iodide; ¹H NMR (DMSO-d₆, 500 MHz) δ 7.87 (s, 8H), 4.72 (t, J=5.0 Hz, 4H), 3.97 (t, J=5.0 Hz, 4H), 3.37 (s, 6H); ¹³C NMR (DMSO-d₆) δ 164.0, 135.9, 129.4, 124.3, 72.1, 63.3, 52.7. The crude ammonium iodide was treated with hydrazine as previously described to give the crude aminooxy product. Purification was accomplished using reverse phase HPLC (Atlantis C₁₈ column, gradient elution 100% H₂O to 100% CH₃CN over 10 min at a flow rate of 15 mL/min; tᵣ=2.08 min) to give 6.2 (0.23 g, 65% yield) as a light yellow solid; ¹H NMR (DMSO-d₆, 500 MHz) δ 6.24 (s, 4H), 3.92 (br s, 4H), 3.57 (br s, 4H), 3.08 (s, 6H); ¹³C NMR (DMSO-d₆) δ 69.1, 63.0, 52.3; HRMS: calcd for C₆H₁₈N₃O₂⁺ (M⁺) requires 164.1394, found 164.1393.

2-(N-methyl-(2-tert-butyldimethylsilyloxyethyl)amino)ethanol (11.1). To a solution of diethanolamine (8.2) (9.0 mL, 78.4 mmol) and Et₃N (2.19 mL, 15.7 mmol) in CH₂Cl₂ (300 mL) at 0° C. was added slowly a solution of tert-butyldimethylchlorosilane (2.35 g, 15.7 mmol) in CH₂Cl₂ (150 mL). On complete addition, the reaction was allowed to warm to room temperature. After 12 h, the reaction mixture was diluted with CH₂Cl₂, transferred to a separatory funnel, and washed successively with saturated NaHCO₃ (3×150 mL) and brine (3×150 mL). The organic layer was dried (Na₂SO₄), filtered and the solvent removed by rotary evaporation. The residue was purified by column chromatography (SiO₂), eluting with a 1:14 mixture of methanol:CH₂Cl₂ (product R_{f} 0.43), to afford silyl ether 11.1 (2.72 g, 75%) as an oil; ¹H NMR (CDCl₃, 500 MHz) δ 3.68 (m, 2H), 3.55 (m, 2H), 2.58 (t, J=5.0 Hz, 4H), 2.31 (s, 3H), 0.85 (s, 9H), 0.02 (s, 6H); ¹³C NMR (CDCl₃) δ 61.4, 59.5, 58.7, 42.8, 26.2, 18.5, −5.11.

2-(N-(2-tert-butyldimethylsilyloxyethyl)-2-hydroxyethylamino)ethanol (11.2). To a solution of triethanolamine (14.2) (5.96 g, 40.0 mmol) and triethylamine (5.6 mL, 40 mmol) in CH₂Cl₂ (10 mL) at 0° C. was added slowly via cannula a solution of tert-butyldimethylsilyl chloride (1.21 g, 8.03 mmol) in CH₂Cl₂ (40 mL). The reaction mixture was stirred 1 h at 0° C. and then allowed to warm to room temperature. After 12 h, the reaction solvent was removed by rotary evaporation. The crude residue was dissolved in EtOAc (40 mL) and washed successively with saturated aq. NaHCO₃ (2 40 mL) and brine (40 mL). The organic layer was dried (Na₂SO₄), filtered, and the solvent was removed by rotary evaporation. The crude product was purified by flash chromatography (SiO₂) using 19:1 CH₂Cl₂:MeOH as eluent (product R_{f}=0.25) to obtain 11.2 (1.90 g, 90%) as a clear oil; IR (neat) 3389, 2928, 2858, 1472 cm⁻¹; ¹HNMR (CDCl₃) δ 3.59 (t, J=5.0 Hz, 2H), 3.50 (t, J=5.5 Hz, 4H), 3.04 (br s, 2H), 2.65 (m, 6H), 0.82 (s, 9H), 0.00 (s, 6H); ¹³C NMR (CDCl₃) δ 62.0, 60.0, 57.1, 56.5, 26.1, 18.5, −5.2.

N,N-bis(2-phthalimidooxyethyl)-2-(tert-butyldimethylsilyloxy)ethyl amine (12.2). To a solution of diol 11.2 (2.38 g, 9.03 mmol), N-hydroxyphthalimide (3.23 g, 19.8 mmol) and triphenylphosphine (5.19 g, 19.8 mmol) in THF (50 mL) at 0° C. was added via syringe diisopropyldiazodicarboxylate (3.93 mL, 19.8 mmol) over 5 min. The reaction was stirred at 0° C. for 0.5 h and then warmed to room temperature. After 12 h, the reaction solvent was removed by rotary evaporation. The orange residue was dissolved in EtOAc (150 mL) and washed successively with saturated aq. NaHCO₃ (3 100 mL), brine (100 mL) and then cold aq. 5% HCl (2 120 mL). The organic layer was stored at 0° C. for 2 h to effect precipitation of the product as its hydrochloride salt. The solids were collected by filtration and washed with cold EtOAc. The collected hydrochloride salt was suspended in a 1:1 mixture of EtOAc:saturated aq. NaHCO₃ and stirred for 0.5 h. The organic layer was separated and washed successively with saturated aq. NaHCO₃, brine, and then dried (Na₂SO₄). The solvent was removed by rotary evaporation and the crude product was purified by column chromatography (SiO₂) eluting with a 2:1 mixture of hexane:EtOAc (product R_{f}=0.23) to obtain 12.2 (4.10 g, 82%) as a light yellow solid; Mp=56-58° C.; ¹H NMR(CDCl₃) δ 7.78 (m, 4H), 7.70 (m, 4H), 4.32(t, J=6 Hz, 4H), 3.71 (t, J=6.0 Hz, 2H), 3.16 (t, J=6.0 Hz, 4H), 2.86 (t, J=6.0 Hz, 2H), 0.84 (s, 9H), 0.02 (s, 6H); ¹³C NMR (CDCl₃) δ 163.6, 134.6, 129.2, 123.7, 62.3, 56.9, 53.4, 26.1, 18.5, −5.2.

2-(N-methyl-(2-phthalimidooxyethyl)amino)ethanol (13.1). Using the Mitsunobu procedure described above for the synthesis of 6.1, silyl ether 11.1 (1.0 g, 4.1 mmol) was transformed into the corresponding mono-phthalimide product. After work-up, the crude mono-phthalimide was dissolved in EtOAc (20 mL) and treated with cold 5% aq. HCl (20 mL). After stirring vigorously for 20 min at room temperature, the layers were separated. The aqueous layer then was extracted with additional portions of Et₂O (3 30 mL), made slightly alkaline by addition of saturated aq. NaHCO₃, and extracted with CHCl₃ (3 50 mL). The combined chloroform extract was dried (Na₂SO₄) and the solvent was removed by rotary evaporation to give 13.1 (0.80 g, 71%) as an oil; ¹H NMR (CDCl₃, 500 MHz) δ 7.73-7.76 (m, 2H), 7.67-7.69 (m, 2H), 4.24-4.26 (m, 2H), 3.54-3.56 (m, 2H), 2.80-2.82 (m, 2H) 2.57-2.59 (m, 2H), 2.32 (s, 3H); ¹³C NMR (CDCl₃) δ 163.7, 134.7, 129.0, 123.7, 76.4, 59.3, 58.8, 55.7, 42.2.

2-(N,N-bis(2-phthalimidooxyethyl)amino)ethanol (13.2). To a solution of bis-phthalimide 12.2 (2.77 g, 5.00 mmol) in THF (15 mL) at 0° C. was added a mixture of HF (2 mL of 48% aq. HF) in THF (14 mL) over 2 min. The reaction mixture was stirred at 0° C. for 1 h before warming to room temperature. After 12 h, the solvents were removed by rotary evaporation. The crude residue was dissolved in EtOAc (100 mL) and washed successively with saturated aq. NaHCO₃ (3 100 mL), brine (100 mL) and then dried (Na₂SO₄). The solvent was removed by rotary evaporation and the crude product was recrystallized in EtOAc to yield 13.2 (1.88 g, 86%) as a light yellow solid; Mp=136-138° C.; IR 3066, 1731 cm⁻¹; ¹H NMR (CDCl₃) δ 7.80 (m 4H), 7.72 (m 4H), 4.36 (t, J=5.0 Hz, 4H), 3.65 (t, J=5.0 Hz, 2H), 3.13 (t, J=5.0 Hz, 4H), 2.91 (t, J=5.0 Hz, 2H) ppm; ¹³C NMR (CDCl₃) δ 163.7, 134.7, 129.1, 123.8, 77.4, 59.4, 57.6, 53.0 ppm; Anal. calcd for C₂₂H₂₁N₃O₇: C, 60.13; H, 4.82; N, 9.56; Found: C, 59.87; H, 4.82; N 9.49.

N-(2-aminooxyethyl)-N-(2-hydroxyethyl)-N,N-dimethylammonium iodide (7.1). In a sealed tube, mono-phthalimide 13.1 (0.60 g, 2.28 mmol) was dissolved in iodomethane (10 mL). The reaction mixture was degassed using a stream of N₂ before sealing the tube. The reaction was heated to 50° C. After 3 h at 50° C., the reaction was cooled and the tube opened. The iodomethane was evaporated to afford the corresponding ammonium iodide which was used in the next step without further purification; ¹H NMR (DMSO-d₆, 500 MHz) δ 7.88 (s, 4H), 5.29 (br s, 1H), 4.67 (br s, 2H), 3.85 (t, J=5.0 Hz, 4H), 3.56 (br s, 2H), 3.24 (s, 6H); ¹³C NMR (DMSO-d₆) δ 164.0, 135.9, 129.5, 124.3, 72.2, 67.0, 63.2, 55.9, 52.6.

To a solution of the crude ammonium iodide in EtOH (5 mL) at room temperature was added hydrazine monohydrate (0.86 mL, 11.3 mmol). After stirring 12 h at room temperature, the solvents were removed by rotary evaporation. The residue was purified using reverse phase HPLC (Atlantis C₁₈ column, gradient elution 100% H₂O to 100% CH₃CN over 10 min at a flow rate of 15 mL/min) to give 7.1 (0.40 g, 65%); ¹H NMR (DMSO-d₆, 500 MHz) δ 6.24 (s, 2H), 5.24 (br s, 1H), 3.92 (br s, 2H), 3.82 (br s, 2H) 3.58 (br s, 2H), 3.43 (br s, 2H), 3.09 (s, 6H); ¹³C NMR (DMSO-d₆) δ 69.2, 66.7, 63.1, 55.9, 52.4. HRMS: calcd for C₆H₁₇N₂O₂⁺ (M⁺) requires 149.1285, found 149.1285.

N,N-bis(2-aminooxyethyl)-N-(2-hydroxyethyl)-N-methylammonium iodide (7.2) In a sealed tube, bis-phthalimide 13.2 (0.88 g, 2.0 mmol) was dissolved in iodomethane (4 mL). The reaction mixture was degassed using a stream of N₂ before sealing the tube. The reaction was heated to 60° C. After 3 h at 60° C., the reaction was cooled and the tube opened. The iodomethane was evaporated (Caution: fume hood required) to afford the corresponding ammonium iodide (1.14 g, 98%), which was used in the next step without further purification; Mp=201° C. (dec); IR 3256, 2921, 1583 cm⁻¹; ¹H NMR (DMSO-d₆) δ 7.87 (s, 8H), 5.36 (t, J=4.5 Hz, 1H), 4.74 (t, J=4.5 Hz, 4H), 4.06 (s, 4H), 3.92 (s, 2H), 3.78 (t, J=4.5 Hz, 2H), 3.42 (s, 3H); ¹³C NMR (DMSO-d₆) δ 164.0, 135.9, 129.4, 124.4, 72.1, 65.5, 61.7, 55.7, 50.7.

To a solution of the ammonium iodide (1.05 g, 1.8 mmol) in a 19:1 mixture of EtOH:H₂O (8 mL) at room temperature was added via syringe hydrazine monohydrate (0.20 mL, 4.1 mmol). After stirring 12 h at room temperature, the solvents were removed by rotary evaporation. The residue was dissolved in H₂O (100 mL) and extracted with EtOAc (3 100 mL). The aqueous layer then was concentrated to ˜2 mL and loaded onto HPLC (reverse phase, C₁₈) using H₂O as the eluent to obtain 7.2 (0.4 g, 70%); ¹H NMR (DMSO-d₆) δ 6.29 (s, br, 4H), 5.26 (s, 1H), 3.97 (s, br, 4H), 3.87 (s, 2H), 3.69 (m, 4H), 3.54 (t, J=5.0 Hz, 2H), 3.16 (s, 3H); ¹³C NMR (DMSO-d₆) δ 69.1, 64.9, 61.5, 55.7, 50.4; Anal. calcd for C₇H₂₀IN₃O₃: C, 26.18; H, 6.28; N, 13.08. Found: C, 26.22; H, 6.14; N 12.73.

### EXAMPLE 2

### Release from Nanoparticles on Warming

NP Formation. Iron oxide nanoparticles (NP) were made according to the procedure described in Mikhaylova et al. Langmuir 2004, 20, 2472-2477.

NP Coating. NPs (3 mg) were suspended in water (5 mL, Millipore, ultrapure) and sonicated 15 min. To the suspension was added N,N-bis-(2-aminooxyethyl)-N,N-dimethylammonium iodide (1, 50 mg) and water (5 mL). The reaction mixture stirred at room temperature. After 12 h, the coated NPs were separated magnetically and washed with water. The washing procedure was repeated five times and then the coated NPs were isolated by freeze drying to obtain NP.1.

NP Loading with FITC2. FITC2 was prepared according to the method described by Tre'visiol et al. European Journal of Organic Chemistry 2000, 1, 211-217. To an aqueous suspension of NP.1 (5 mL, NP.1 concentration at 0.8 mg/mL) was added FITC2 (15 mg). The mixture was vigorously mixed 15 min. and then water (5 mL) was added. After stirring at rt 12 h, the NP.1-FITC2 particles were magnetically separated and washed according to the procedure described above using methanol. The separated particles then were isolated after freeze drying to give NP.1-FITC2.

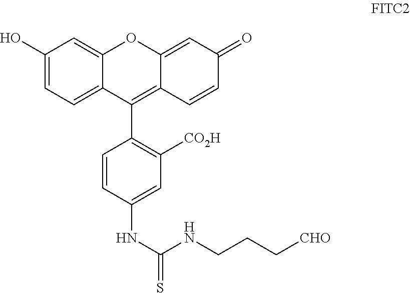

UV-Visible spectroscopy measurements were taken of NP, NP.1, NP.1-FITC2 and FITC2 at concentrations of 0.025 mg/mL. As a control, unmodified NP were mixed with FITC2. The UV data indicates the aldehyde substrate FITC2 is bound to the nanoparticle only when compound 1 is present. FITC2 was not bound unless compound 1 was loaded onto the NP first, implicating the oxime ether linkage as the tethering functionality.

Heat-Induced Release. The NP.1-FITC2 particles were placed in a 15 mL glass vial and water (10 mL) was added. The suspension was vortex mixed 15 min and then heated at 43° C. for 40 min using an oil bath. The particles were sedimented by centrifugation and the supernatant collected and analyzed by UV-Vis spectroscopy. The data shows that the NPs no longer contained FITC2 after the heating experiment. In contrast, the FITC2 was released from the NPs and observed in the supernatant after separation of the NPs. Thus, a compound can be bound to a magnetic nanoparticle, and the compound is not released until sufficiently warmed, e.g., by an externally-located source, thereby allowing for targeted delivery of the compound.

### EXAMPLE 3

### Release of Conjugates from Nanoparticles on Warming

Described herein is an experiment and mass spectral data demonstrating the release of the oxime ether conjugate from a nanoparticle preparation on warming. 4-HNE was used as the representative drug molecule. Oxime ether derivatives (e.g., O-alkyl oximes) themselves are important drugs or as analogs and the O-alkyl oxime functionality is present in many drugs and drug candidates (see, e.g., Choong et al., J Org. Chem., 64, 6528-6529 (1999); hereinafter Choong). Further, FIG. 1 of Choong depicts two oxime ether drugs. The oxime ether group is hydrolized to unmask the actual drug. Thus, the release of the instant oxime ether derivatives can be thought of, in some embodiments, as the release of prodrugs.

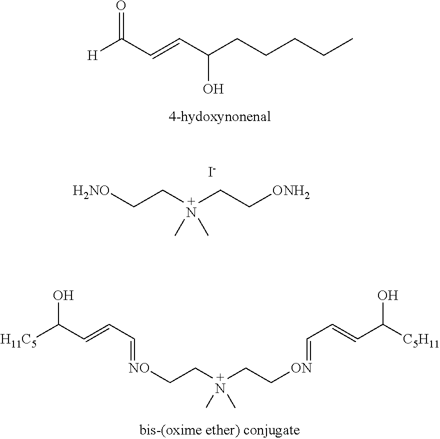

NP.1 (nanoparticles coated with N,N-bis-(2-aminooxyethyl)-N,N-dimethylammonium iodide, compound 1) were loaded with 4-hydroxynonenal (4-HNE), a product of lipid peroxidation in cells. The loaded particles then were washed several times to remove any trace of unreacted 4-HNE. To demonstrate that heat can induce release of the corresponding oxime ether conjugate (bound to the surface of the NPs), a suspension of the multiply washed NP.1.4-HNE particles was heated to 40° C. The supernatant then was analyzed by HRMS for the presence of the bis-oxime ether conjugate. The data clearly show release of the bis-conjugate from the nanoparticle preparation.

Experimental Procedure for the 4-HNE experiment. To a suspension of NP.1 (1 mg, contains ˜0.4 mg of 1 according to dry weight measurements) in methanol (0.05 mL) was added a solution of (±)-(2E)-4-hydroxy-2-nonenal (4-HNE) (49 μL of a 15 μg/mL ethanol solution; roughly a 2:1 4-HNE:1 molar ratio). The particles were collected after 16 h by magnetic separation (sedimentation assisted by placing a magnet under the reaction vial followed by removal of the supernatant). The NP.1.4-HNE particles were then suspended in 1:1 water:methanol and vortex mixed followed by magnetic separation. This procedure was performed an additional 5 times and one final time using only water.

To induce heat release, the NP.1.4-HNE particles were suspended in water (1 mL) and warmed by submersing the suspension in an oil bath heated to 40° C. After 45 mins, the suspension was cooled to room temperature and methanol (1 mL) was added. The mixture was placed in a centrifuge to pellet the nanoparticles. The supernatant was removed and submitted for analysis via HRMS.

A. Reference HRMS—bis-(oxime ether) conjugate of 4-HNE and 1. The bis-oxime ether conjugate was prepared by simple mixing of the two compounds and then analyzed by HRMS (nanospray FTMS 10×dil in MeCN). The MS2 spectrum shows that mono-4HNE adduct is not formed by fragmentation.

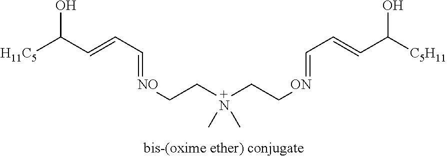

B. Final supernatant of 4-HNE experiment. The NP.1.4-HNE particles were suspended in 1:1 H₂O:MeOH and vigorously vortex mixed followed by separation of the particles from the wash solution using a magnet. This procedure was performed a total of six times followed by a final rinse using only H₂O. The particles then were suspended in H₂O and the suspension was warmed to 40° C. After 45 minutes, the particles were sedimented by centrifugation and the supernatant from this heat-release experiment was analyzed by HRMS. The bis-(oxime ether) derivative was clearly detected in the spectra.

C. Final supernatant of BLANK. NP.1 particles (blank—no 4-HNE) were washed in the same manner as described above and then heated at the same temperature for the same time. Analysis by HRMS did not show any signal at 440.35 that would correspond to the bis adduct.

### EXAMPLE 4

### Release of Oxime Ether Conjugates from Nanoparticles using an Alternating Electromagnetic Field (AEM); AEM Field-Mediated Release of Doxorubicin to Cancer Cells

Doxorubicin (brand names Adriamycin®, Rubex®) is an example of an anthracycline antibiotic used as a chemotherapy drug to treat cancers, such as bladder, breast, head and neck, liver, lung, ovary, pancreas, prostate, thyroid and uterus cancer. It is given by intraveneous injection (IV), and there is no pill form of doxorubicin. A major problem associated with doxorubicin treatment is toxicity, particularly liver and cardiotoxicity (see, e.g., Lebrecht et al., Int. J. Cancer., 120, 927-934 (2007)). Doxorubicin is a vesicant and will cause extensive tissue damage and blistering if it escapes from the vein. Thus there have been many efforts to develop safe delivery systems to precisely target doxorubicin to cancer sites (Yavlovich et al., Biochim. Biophys. Acta (2010), Burke et al., J. Med. Chem., 47, 1193-1206 (2004), and Burkhart et al., Mol. Cancer Ther., 3, 1593-1604 (2004)). Demonstrated herein, with an in vitro model, is the effective use of iron oxide nanoparticles to deliver doxorubicin to cancer cells in response to irradiation with an alternating electromagnetic (AEM) field.

Doxorubicin-loaded iron oxide nanoparticles were prepared using two different methods (see Scheme D below): (a) In the stepwise method, iron oxide NPs were first coated with an aminooxy compound (in this case, an aminooxy alcohol 3), and then reacted with a keto-drug (doxorubicin). The drug attaches to the NPs via oximation of the C(9)-hydroxyacetyl ketone group to afford the loaded NPs sNP.AO.Dox. (b) In the direct method, iron oxide NPs were directly treated with the C(9)-hydroxyacetyl oxime ether conjugate of aminooxy compound 3 and doxorubicin (Dox.AO) to afford the loaded NPs dNP.AO.Dox.

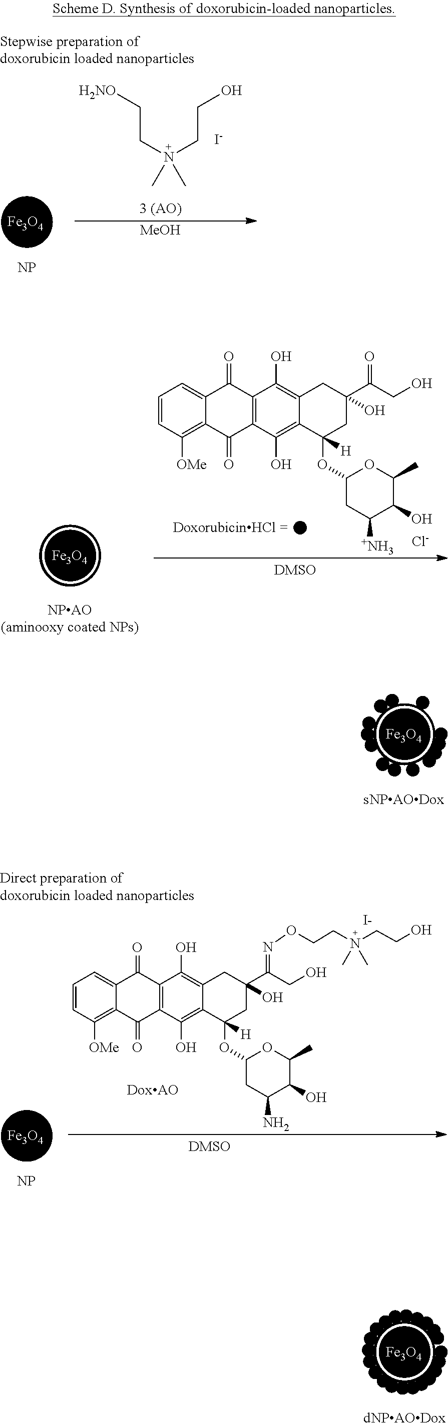

Breast cancer MCF-7 cells were treated with the resultant Dox-NP formulations, and the treated cells then were either allowed to incubate or briefly exposed to an AEM field to induce drug release from the Dox-NP formulations. The results are summarized in Table 1 below.

There was no increase in cell death in the absence of an AEM field (Table 1, entries 6, 8, 10 and 12), demonstrating that doxorubicin-nanoparticle formulations can serve as inactive pro-drugs of doxorubicin. This is an important feature for use as a drug delivery system: Attachment of a drug to an iron oxide NP via an oxime ether linkage can render the drug inactive. In contrast, when exposed to an AEM field, all NP.Dox formulations released doxorubicin, either as the free drug (Table 1, entry 5) or as the oxime ether conjugate (Table 1, entries 7, 9, and 11), to cause an increase in cell death relative to untreated cells. The extent of cell death was dependent both on dose (0.5 mg dose of a NP formulation was more effective than the 0.25 mg dose, compare entries 7 and 9) and on the method of doxorubicin loading (compare entries 5, 7, and 11). Doxorubicin does not adhere effectively to unfunctionalized iron oxide nanoparticles (see NP.Dox loading, entry 5). In contrast, the cationic aminooxy compound (AO) increased the overall attachment of drug to NPs, either by first attaching the AO to NPs (stepwise synthesis) or by first attaching AO to the drug (direct synthesis). Accordingly, drugs can be effectively bound to iron oxide nanoparticles by oximation with cationic aminooxy compounds (AO) after NP loading of the AO or by NP loading of the corresponding AO-drug oximation product.

AEM field study. Results of MCF-7 cells treated with nanoparticle-doxorubicin formulations followed by alternating electromagnetic (AEM) field exposure (15 mins, 350 A, 203 KHz) were obtained. Cells plated in 30 mm dishes were treated with NP formulations at a 0.5 mg Fe₃O₄ dose unless otherwise specified. Doxorubicin loadings per mg NP formulation were as follows: dNP.AO.Dox (0.64 mg Dox/mg), sNP.AO.Dox (0.43 mg Dox/mg), NP.Dox (0.05 mg Dox/mg). NP.AO (nanoparticle-aminooxy only) and Dox (doxorubicin.HCl, 0.25 mg dose) serve as negative and positive control experiments, respectively. The results are summarized in Table 1.

AEM field irradiation itself did not cause the noted increases in cell death (see control, entry 2), and AEM field irradiation of cells treated with NP.AO particles (no doxorubicin conjugates attached) did not cause an increase in cell death (see control, enty 3). The results tabulated in Table 1 show the effectiveness of AEM field irradiation on drug delivery. Only when the s- and dNP.AO.Dox formulations (prodrug formulations) were irradiated by an AEM field did cell death occur to any appreciable extent, similar to direct administration of doxorubicin (entry 4).

TABLE 1
Results of AEM field-induced release
of doxorubicin from NP formulations.

| | | | ~Dose^{a} | | |
|---|---|---|---|---|---|
| | | Dose (mg) | (mg) of | AEM field | MCF-7 |
| Entry | Treatment | of Fe₃O₄ | Dox admin. | exposure | cell death^{b} |
| 1 | Cells only | 0 | 0 | No | No |
| 2 | Cells only | 0 | 0 | Yes | No |
| 3 | NP•AO | 0.5 | 0 | Yes | No |
| 4 | Dox | 0 | 0.3 | No | Yes |
| | | | | | (major) |
| 5 | NP•Dox | 0.5 | ~0.025 | Yes | Yes |
| | | | | | (minor) |
| 6 | NP•Dox | 0.5 | ~0.025 | No | No |
| 7 | dNP•AO•Dox | 0.5 | ~0.32 | Yes | Yes |
| | | | | | (major) |
| 8 | dNP•AO•Dox | 0.5 | ~0.32 | No | No |
| 9 | dNP•AO•Dox | 0.25 | ~0.16 | Yes | Yes |
| | | | | | (minor) |
| 10 | dNP•AO•Dox | 0.25 | ~0.16 | No | No |
| 11 | sNP•AO•Dox | 0.5 | ~0.22 | Yes | Yes |
| | | | | | (major) |
| 12 | sNP•AO•Dox | 0.5 | ~0.22 | No | No |

^{a}Assuming 100% release of the NP payload on irradiation.
^{b}Relative to the ‘cells only’ control.

### Experimental Procedures

Synthesis of conjugate Dox.AO. To a solution of doxorubicin.HCl (9.0 mg, 0.015 mmol) in anhydrous methanol (7 mL) at room temperature was added N-(2-hydroxyethyl)-N,N-dimethyl-2-aminooxyethylammonium iodide (AO) (21 mg, 0.077 mmol) and trifluoroacetic acid (0.1 mL). After stirring the reaction mixture at room temperature for 96 h, the solvent was removed under reduced pressure. Acetonitrile (5 mL) was added to the residue and the suspension was sonicated 1 min at room temperature and then heated at 40° C. for 5 min. The suspension was then cooled to −20° C. After storing 18 h at −20° C., the supernatant was decanted and the remaining precipitate (red solid) was dried under reduced pressure to afford Dox.AO (11.3 mg, 91%); HRMS, calculated for C₃₃H₄₄N₃O₁₂⁺ (M⁺), 674.2920; found, 674.2926.

TABLE 2
Comparison of ¹³C NMR C═O shifts for Dox and Dox•AO.

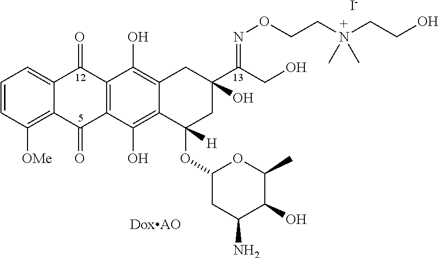

| Compound | Carbon Position | δ (ppm)^{a} |
|---|---|---|
| Dox | C-5 | 186.9 |
| Dox•AO | C-5 | 186.8 |
| Dox | C-12 | 186.6 |
| Dox•AO | C-12 | 186.4 |
| Dox | C-13 | 213.2 |
| Dox•AO | C-13 | 162.6 |

^{a}Spectra taken in CD₃OD; to compare these Dox shifts to those reported in the literature, see: Krüger et al., Chem. Pharm. Bull. 1997, 45, 399-401.

Method for direct preparation of dNP.AO.Dox. Fe₃O₄ nanoparticles (the NPs were prepared according to the method described by Mikhaylova et al. Langmuir 2004, 20, 2472-2477) (3.8 mg) were suspended in anhydrous DMSO (1.5 mL) at room temperature followed by sonication (15 min, room temperature) using an Ultra Sonicator™ water bath (Laboratory Supplies Co., Inc., Ithica, N.Y.). Within 5 minutes of sonication, a solution of Dox.AO (15 mg) in anhydrous DMSO (1.5 mL) was added to the NP suspension. The reaction suspension was sonicated (15 min) at room temperature and then stirred 12 h. The resultant dNP.AO.Dox particles were magnetically sedimented to facilitate removal of the supernatant. The particles then were washed with DMSO (1.5 mL, 3×). The washed particles were dried under reduced pressure to afford dNP.AO.Dox (6.75 mg; loading: 0.77 mg Dox.AO/mg NP≅0.64 Dox/mg NP).

Preparation of NP.AO. A suspension of Fe₃O₄ nanoparticles (5.5 mg) in methanol (1.5 mL) at room temperature was sonicated (15 min) followed by addition of a solution of N-(2-hydroxyethyl)-N,N-dimethyl-2-aminooxyethylammonium iodide (AO) (30 mg) in methanol (1.5 mL). The reaction suspension was sonicated (10 min) at room temperature and then stirred 12 h. The resultant NP.AO particles were magnetically sedimented to facilitate removal of the supernatant. The particles then were washed with methanol (1.5 mL, 2×). The washed particles were dried under reduced pressure to afford NP.AO (7.42 mg; loading: 0.35 mg AO/mg NP).

Method for stepwise preparation of sNP.AO.Dox. A suspension of NP.AO (3.0 mg) in anhydrous DMSO (1.5 mL) at room temperature was sonicated (15 min). Within 5 minutes of sonication, a solution of doxorubicin.HCl (5.5 mg) in anhydrous DMSO (1.5 mL) was added. The reaction suspension then was sonicated (15 min) at room temperature and stirred at room temperature an additional 12 h. The resultant sNP.AO.Dox particles were magnetically sedimented to facilitate removal of the supernatant. The particles then were washed with DMSO (1.5 mL, 3×). The washed particles were dried under reduced pressure to afford sNP.AO.Dox (4.3 mg; loading: 0.43 mg Dox/mg NP).

Preparation of NP.Dox. A suspension of Fe₃O₄ nanoparticles (5.0 mg) in anhydrous DMSO (1.5 mL) at room temperature was sonicated (15 min) followed by addition of a solution of doxorubicin.HCl (10 mg) in anhydrous DMSO (1.5 mL) This reaction suspension was sonicated (15 min) and then stirred at room temperature for 12 h. The resultant NP.Dox particles were magnetically sedimented to facilitate removal of the supernatant. The particles then were washed with DMSO (1.5 mL, 3×). The washed particles were dried under reduced pressure to afford NP.Dox (5.3 mg, loading: 0.05 mg Dox/mg NP).

Cell culture toxicity study. Human breast cancer cells (MCF-7) were purchased from American Type Culture Collection (VA, USA). Cells were plated in 30 mm dishes at ˜4˜10⁵ cells/dish, and then grown up to 50-60% confluency in DMEM, 1% Pennstrep (Mediatech, Inc, VA) and 10% FBS (Valley Biomedical, Winchester, Va.).

Alternating electromagnetic field-induced drug release in MCF-7 cells. Stock solutions of the nanoparticle formulations NP.AO, NP.Dox, dNP.AO.Dox and sNP.AO.Dox were prepared in PBS-1× buffer solution at a uniform Fe₃O₄ concentration of 1 mg/mL To the MCF-7 cells maintained at 37.5° C. were added specific doses (either 0.25 mg or 0.5 mg) of NP formulations and then followed by exposure to an alternating electromagnetic field (AEM field). Treatment groups:

dNP.AO.Dox with and without AEM field, 0.25 and 0.5 mg doses

sNP.AO.Dox with and without AEM field, 0.5 mg dose

NP.Dox with and without AEM field, 0.5 mg dose

NP.AO with and without AEM field, 0.5 mg dose

Cells with and without AEM field exposure and cells treated with doxorubicin.HCl (0.3 mg) without AEM field exposure served as control experiments.

Each measured amount of NP formulation was diluted to 2 mL by adding DMEM containing 10% FBS. After growth medium was taken from the cells, the treatment solution was added to the cells in the 30 mm dish. The cells then were incubated at 37.5° C. After 1 h incubation, those cells planned for AEM field irradiation were exposed to an AEM field generated by an EASYHEAT™ 8310LI solid state induction power supply (Ameritherm, Inc., 5 turn coil with ID: 5.0 cm and OD: 6.5 cm). In each case, AEM field irradiation was performed for 10 min at a frequency of 203 kHz and power of 350 A. After irradiation, the cells were incubated at 37.5° C.

After incubation for a total of 48 h, analyses of dead (floating) cells were performed using a hemocytometer. The results are expressed in Table 1 above.

### EXAMPLE 5

### Release of Oxime Ether Conjugates from Nanoparticles using an Alternating Electromagnetic Field (AEM); Aminooxy Structure and NP-Binding and AEM Field-Mediated Release Properties

Cationic aminooxy compounds can adhere tightly to anionic iron oxide nanoparticles; but not too tightly since AEM field exposure, and the accompanying heat generated on AEM field exposure, causes release of the aminooxy compounds and attached drug conjugates from the nanoparticle (NP) surface. Release from the NPs is an important event for conversion of the prodrug (NP.AO.Drug) into the active drug (AO.Drug→AO+Drug). It has been determined, as described herein, that the molecular structure of the ammonium aminooxy compounds strongly influences binding and release properties. Accordingly, as described herein, this class of aminooxy compounds can be structurally tailored to adjust drug binding and/or release properties.

The fluorophore FITC-CHO was attached to iron oxide nanoparticles by reaction with NP-bound aminooxy compounds (previously described compounds 6.1, 7.1 and the diol analog of 7.1) to show the influence of resident hydroxyl functionality on the binding and release properties of derived iron oxide formulations. Scheme E below depicts the fluorophore aldehyde and the three oxime ether conjugates selected for this study. Using the stepwise synthesis method, FITC-CHO was mixed with NP-AO particles derived from the three aminooxy compounds to give the NP.(AO).FITC fluorophore formulations. These formulations then were washed (supernatants collected) and then irradiated with an AEM field to induce AO.FITC conjugate release (supernatant collected). The relative binding and release properties were measured by fluorescence spectroscopy on the supernatant solutions after magnetic separation of the nanoparticles and/or NP.(AO).FITC particles.

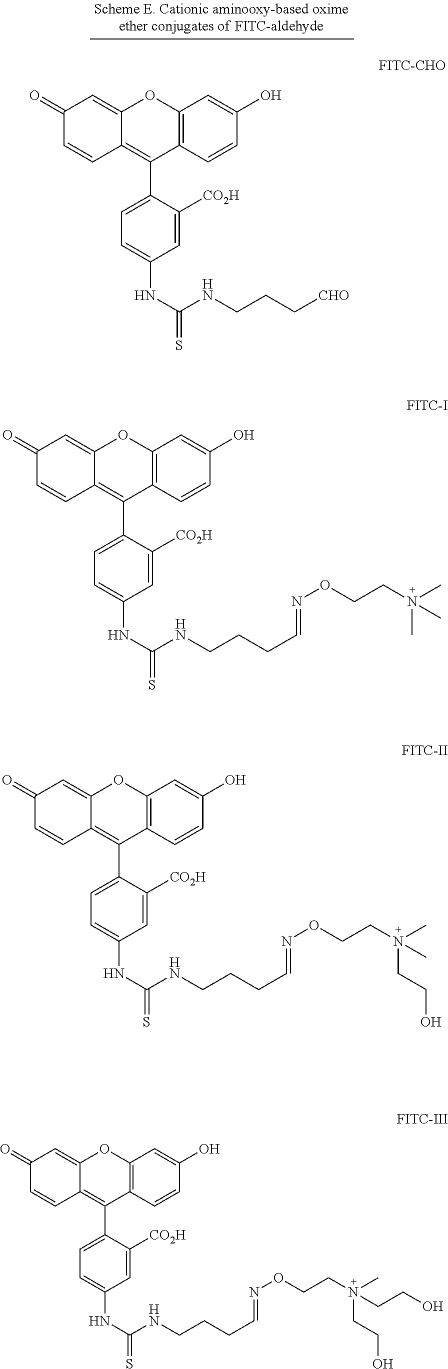

Integration of the supernatant fluorescence after NP washing and after AEM field irradiation is indicative of the binding to the nanoparticles and the release of FITC into the supernatant. The negative control (FITC-CHO reacted directly with nanoparticles, no AO coating) shows an almost complete loss of fluorescence throughout the washes and no recovery of fluorescence after exposure to an AEM field; thus, no retention of FITC-CHO on NPs (HPLC traces for free FITC-CHO show 3 peaks, two of which result from isomeric forms of the fluorophore and one is a constitutional isomer).

Under identical concentration, wash and irradiation conditions, the release of conjugate FITC-III in response to AEM field irradiation is seen. In contrast to the negative control, considerable FITC-conjugate is released into solution in response to the irradiation. In addition, release of a small amount of FITC-CHO, presumably bound to the cationic aminooxy coating in a non-covalent manner, is also observed.

The NP.AO-FITC-II and NP.AO-FITC-III formulations showed an increase in supernatant fluorescence after AEM field exposure, indicating release of the conjugates in response to the external stimulus. These results show the importance of the aminooxy coating as well as the role of hydroxyl functionality in maintaining the FITC-CHO conjugate (drug surrogate) on the iron oxide nanoparticles until irradiated. Modulating the extent of hydroxyl substitution on the aminooxy layer thus can be expected to influence loading and release.

### Experimental Procedures

General method for synthesis and AEM field irradiation of NP.(AO).FITC particles. Aqueous solutions (0.1 M) of ammonium aminooxy compound were added to suspensions of iron oxide nanoparticles (3 mg) in vials (5 μmoles of aminooxy compound/mg of nanoparticles). The vials were briefly sonicated (15 minutes) and then vortex mixed for 45 minutes at room temperature. The resultant NP.AO nanoparticles were magnetically separated from the supernatant solution, washed with water to remove unbound ammonium aminooxy salt, and freeze dried.

To a suspension of the NP.AO (3 mg) in methanol at room temperature was added a solution of FITC-CHO in methanol (0.01M, 50 μL/mg of NP.AO). The reaction mixture was vortex mixed 2 hours at room temperature. The particles then were magnetically separated, the supernatant was removed and analyzed. The particles were washed twice with methanol (3× reaction volume) and the supernatants collected each time and analyzed. Water was then added (50 μL/mg NP.AO.FITC), an aliquot was collected for analysis, and the aqueous suspensions then were placed in an Ambrell Easy Heat LI alternating electromagnetic field at 201.4 A and 203 kHz for 30 minutes. After irradiation, the particles were removed by magnetic separation and an aliquot of the final supernatant was collected for analysis.

The collected aliquots, both pre- and post-AEM field exposure, were identically diluted and their fluorescence was measured during reverse phase HPLC using a Waters Atlantis C18 column (5 μm, 3.9×150 mm) on an Agilent 1100 series HPLC. The total run-time was 45 minutes with the following gradient: (% line B, elapsed time in minutes) 0,0; 0,3; 60,10; 80,25; 100,30; 100,35; 0,45 where the A line was 5 mM ammonium acetate (pH 6) and the B line was 75% v/v aqueous acetonitrile. Excitation-emission spectra (EES) of FITC-CHO, FITC-I, and FITC-II were acquired on a PerkinElmer LS55 fluorescence spectrometer in order to determine the excitation wavelengths that would yield maximum emission for each adduct. The excitation wavelengths selected for the fluorescence measurements were chosen based on these EES. The fluorescence excitation beam-width of the Agilent instrument was 20 nm.

TABLE 3

| Sample | Excitation λ at max emission | Emission λ |
|---|---|---|
| FITC-I | 520 | 521 |
| FITC-II | 514 | 520 |
| FITC-III | 517 | 520 |
| FITC-CHO | 512 | 520 |

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A magnetic nanoparticle comprising a core, wherein the nanoparticle comprises at least one therapeutic agent linked to the core via a hydrazone linkage or via an oxime ether linkage.

2. A magnetic nanoparticle comprising a core, wherein the nanoparticle comprises reactive hydrazine or aminooxy groups linked to the core of the nanoparticle.

3. The nanoparticle of claim 1, wherein at least one therapeutic agent is a chemotherapeutic agent, an antibiotic agent, an antifungal agent, an antiparasitic agent or an antiviral agent.

4. The nanoparticle of claim 1, wherein at least one therapeutic agent is an anthracycline antibiotic.

5. The nanoparticle of claim 1, wherein at least one therapeutic agent is doxorubicin.

6. The nanoparticle of claim 1, wherein at least one therapeutic agent is linked to the core via a hydrazone linkage.

7. The nanoparticle of claim 1, wherein at least one therapeutic agent is linked to the core via an oxime ether linkage.

8. The nanoparticle of claim 1, wherein the nanoparticle comprises reactive hydrazine groups linked to the core of the nanoparticle.

9. The nanoparticle of claim 1, wherein the nanoparticle comprises reactive aminooxy groups linked to the core of the nanoparticle.

10. The nanoparticle of claim 1, wherein the core of the nanoparticle is about 5-50 nm in diameter.

11. The nanoparticle of claim 1, wherein the nanoparticle has an iron oxide core.

12. The nanoparticle of claim 1, wherein the nanoparticle further comprises a targeting element.

13. The nanoparticle of claim 1, wherein the nanoparticle further comprises a carbohydrate or carbohydrate fragment.

14. A method of making a nanoparticle, comprising combining a magnetic nanoparticle having a core with an aminooxy agent to make an iron oxide nanoparticle that comprises reactive aminooxy groups linked to the core of the nanoparticle.

15. The method of claim 14, further comprising reacting the nanoparticle that comprises the aminooxy groups with at least one agent to make a nanoparticle that comprises at least one agent linked to the core of the nanoparticle via an oxime ether linkage.

16. The method of claim 15, wherein at least one agent is a therapeutic agent.

17. The method of claim 14, wherein the aminooxy agent is an aminooxy alcohol.

18. The method of claim 14, wherein the aminooxy agent is an agent having the formula: 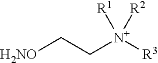
wherein R¹, R², and R³ are each individually alkyl optionally substituted with one or more —OH, —CF₃, —N⁺, or —ONH₂ groups.

19. The method of claim 18, wherein the aminooxy agent is a compound selected from

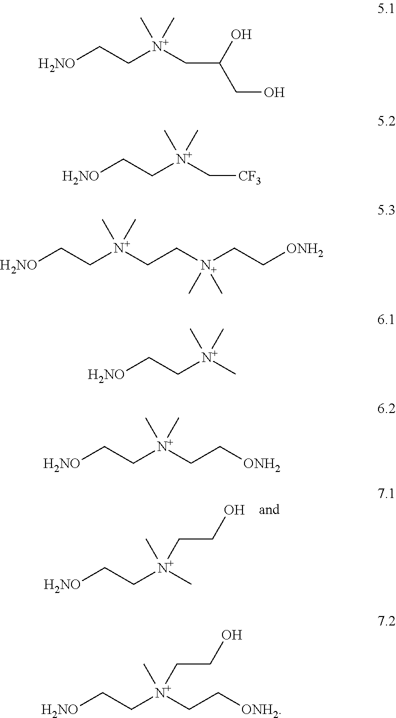

20. A method of making a nanoparticle, comprising combining a magnetic nanoparticle having a core with an oxime ether conjugate of an aminooxy agent and a therapeutic agent so as to make a nanoparticle that comprises the therapeutic agent linked to the core via a hydrazone linkage or via an oxime ether linkage.

21. A method for administering a therapeutic agent to a patient, comprising administering the nanoparticle of claim 1 to the patient.

22. The method of claim 21, further comprising magnetically targeting the nanoparticles to a specific location in the patient.

23. The method of claim 21, wherein the nanoparticle comprises a targeting element.

24. The method of claim 21, further comprising delivering a source of heat so as to release the therapeutic agent, or a prodrug of the therapeutic agent, from the nanoparticle.

25. The method of claim 21, further comprising applying an alternating electromagnetic field to the patient to release the therapeutic agent, or a prodrug of the therapeutic agent, from the nanoparticle.

26. A method for separating a compound having a reactive aldehyde or ketone group from a mixture of compounds, comprising:
adding the nanoparticle of claim 2 to the mixture;
allowing the nanoparticle to bind to the compound having a reactive aldehyde or ketone group; and
separating the bound nanoparticle from the mixture.

27. The method of claim 26, further comprising identifying the compound bound to the nanoparticle.

28. A method for administering a therapeutic agent to a patient, comprising:
administering the nanoparticle of claim 2 to the patient;
targeting the nanoparticle to a specific site in the patient's body;
administering a therapeutic agent that comprises an aldehyde or ketone group to the patient;
allowing the nanoparticle and therapeutic agent to bind together; and
applying an alternating electromagnetic field to the specific site in the patient's body to release the therapeutic agent from the nanoparticle.

29. The method of claim 28, wherein the nanoparticle is targeted to the specific site magnetically.

30. The method of claim 28, wherein the nanoparticle comprises a targeting element that targets the nanoparticle to the specific site.

31. A composition comprising a nanoparticle as described in claim 1 and an acceptable carrier.

32. The composition of claim 31, wherein the acceptable carrier is a pharmaceutically acceptable carrier.

33. The composition of claim 31, wherein the composition comprises a first population of nanoparticles that are individually linked via a hydrazone linkage or an oxime ether linkage to a first therapeutic agent and a second population of nanoparticles that are individually linked via a hydrazone linkage or an oxime ether linkage to a second therapeutic agent that is a different therapeutic agent than the first therapeutic agent.

34-37. (canceled)
